(19) United States
(12) Patent Application Publication
(10) Pub. No.: US 2024/0342475 A1
(43) Pub. Date: Oct. 17, 2024

(54) Managing Orthostatic Hypotension with SCS Closed Loop Stimulation

(71) Applicant: Boston Scientific Neuromodulation Corporation, Valencia, CA (US)

(72) Inventors: Lisa Moore, Glendale, CA (US); Raul Serrano Carmona, Venice, CA (US); Tianhe Zhang, Studio City, CA (US)

(21) Appl. No.: 18/627,142

(22) Filed: Apr. 4, 2024

Related U.S. Application Data
(60) Provisional application No. 63/495,449, filed on Apr. 11, 2023.

(51) Int. Cl. A61N 1/36; A61N 1/05; A61N 1/372; A61N 1/375

(52) U.S. Cl. CPC A61N 1/36062; A61N 1/0551; A61N 1/36071; A61N 1/36139; A61N 1/36171; A61N 1/37258; A61N 1/37282; A61N 1/3754

(57) ABSTRACT

Systems and methods for using spinal cord stimulation (SCS) for controlling orthostatic hypotension in a patient are described. Embodiments are configured to monitor for changes in the patient's state and apply stimulation when needed. The change in state may be a change in the patient's inertial state, such as a change in posture, activity, or the like. The change in state may also be indicated based on sensed neural activity. Embodiments provide closed-loop feedback control of the stimulation.

### CROSS REFERENCE TO RELATED APPLICATIONS

This is a non-provisional of U.S. Provisional Patent Application Ser. No. 63/495,449, filed Apr. 11, 2023, to which priority is claimed, and which is incorporated herein by reference.

### FIELD OF THE INVENTION

This application relates to spinal cord stimulation (SCS), and more specifically to closed loop SCS for the management of orthostatic hypotension.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS), which is commonly used to treat pain in a patient's back and/or limbs. SCS can also be used to treat symptoms related to gait, balance, and other autonomic functions in patients with movement disorders such as Parkinson's disease (PD) and multiple system atrophy (MSA).

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application specific and therefore can vary. The conductive case 12, or some conductive portion of the case, can also comprise an electrode (Ec). In an SCS application, the electrode lead(s) are typically implanted in the spinal column proximate to the dura in a patient's spinal cord, preferably spanning left and right of the patient's spinal column. The proximal contacts 21 are tunneled through the patient's tissue to a distant location such as the buttocks where the IPG case 12 is implanted, at which point they are coupled to the lead connectors 22. SCS therapy can relieve symptoms such as chronic back pain. IPG 10 as described should be understood as including External Trial Stimulators (ETSs), which mimic operation of the IPG 10 during trials periods when leads have been implanted in the patient but the IPG 10 has not. Sec, e.g., U.S. Pat. No. 9,259,574 (disclosing an ETS).

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30i), as shown in the example of FIG. 2A. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as an anode (during its first phase 30a), and thus provides pulses which source a positive current of amplitude +I to the tissue. Electrode E2 has been selected as a cathode (again during first phase 30a), and thus provides pulses which sink a corresponding negative current of amplitude −I from the tissue. This is an example of bipolar stimulation, in which the lead includes one anode pole and one cathode pole. Note that more than one electrode on the lead may be selected to act as an anode electrode to form an anode pole at a given time, and more than one electrode may be selected to act as a cathode to form a cathode pole at a given time, as explained further in U.S. Pat. No. 10,881,859. Stimulation provided by the IPG 10 can also be monopolar. In monopolar stimulation, the lead is programmed with a single pole of a given polarity (e.g., a cathode pole), with the conductive case electrode Ec acting as a return (e.g., an anode pole). Again, more than one electrode on the lead may be active to form the pole during monopolar stimulation.

IPG 10 as mentioned includes stimulation circuitry 28 to form prescribed stimulation at a patient's tissue. FIG. 3 shows an example of stimulation circuitry 28, which includes one or more current source circuits and one or more current sink circuits. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is associated with an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient's tissue, R, hopefully with good therapeutic effect. Consistent with the example provided in FIG. 2A, FIG. 3 shows operation during the first phase 30a in which electrode E1 has been selected as an anode electrode to source current I to the tissue R and E2 has been selected as a cathode electrode to sink current from the tissue. Thus PDAC1 and NDAC2 are digitally programmed to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16. Other stimulation circuitries 28 can also be used in the IPG 10, including ones that includes switching matrices between the electrode nodes ci 39 and the N/PDACs. Sec, e.g., 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ci 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as IPG master control circuitry 102 (see FIG. 5), telemetry circuitry (for interfacing off chip with telemetry antennas 27a and/or 27b), circuitry for generating the compliance voltage VH (as explained next), various measurement circuits, etc.

Power for the stimulation circuitry 28 is provided by a compliance voltage VH, as described in further detail in U.S. Patent Application Publications 2013/0289665 and 2018/0071520. The compliance voltage VH may be coupled to the source circuitry (e.g., the PDAC(s)), while ground may be coupled to the sink circuitry (e.g., the NDAC(s)), such that the stimulation circuitry 28 is powered by VH and ground. Other power supply voltages may be used with the PDACs and NDACs, and explained in U.S. Patent Application Publication 2018/0071520, but these aren't shown in FIG. 3 for simplicity.

Preferably, and as described in U.S. Pat. No. 11,040,202, the compliance voltage VH can be produced by a VH regulator 49. VH regulator 49 receives the voltage of the battery 14 (Vbat) and boost this voltage to a higher value required for the compliance voltage VH. VH regulator 49 can comprise an inductor-based boost converter or a capacitor-based charge pump for example. The regulator 49 can vary the value of VH based on measurements taken from the stimulation circuitry 28. As explained in detail in the '202 patent, VH measurement circuitry 51 can be used to measure the voltage drops across the active DACs (e.g., PDAC1 (Vp1) and NDAC2 (Vn2) in the example shown in FIG. 3) in the stimulation circuitry 28. Using such measurements allows VH to be established at an energy-efficient level: high enough to form the prescribed current without loading (i.e., without producing less current that prescribed), yet low enough to not needlessly waste power in the stimulation circuitry 28 when forming the prescribed current. In this respect, VH can be variable, and typically ranges from about 5 to 15 Volts.

The VH measurement circuitry 51 can output an enable signal VH (en1) indicating when VH regulator 49 should increase the level of VH, i.e., when the voltage drops across the active DACs are too low. This enable signal VH (en1) may be processed at logic 53 in conjunction with other signals explained below to determine a master enable signal VH (en) for the VH regulator 49. Logic 53 may be associated with the IPG's control circuitry 102. Master enable signal VH (en) when asserted causes the VH regulator 49 to increase VH (e.g., when the current starts to load). Deasserting VH (en) disable the VH regulator, which allows VH to naturally decrease over time until it needs to be increased again. This feedback generally causes VH to be established at an energy-efficient value appropriate for the current that is being provided by the stimulation circuitry 28.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861. While useful, DC-blocking capacitors 38 are not strictly required in all IPG designs and applications.

Referring again to FIG. 2A, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will (primarily) build up across the DC-blockings capacitors C1 and C2 associated with the electrodes E1 and E2 used to produce the current, giving rise to voltages Vc1 and Vc2 (I=C*dV/dt). During the second pulse phase 30b, when the polarity of the current I is reversed at the selected electrodes E1 and E2, the stored charge on capacitors C1 and C2 is recovered, and thus voltages Vc1 and Vc2 hopefully return to 0V at the end the second pulse phase 30b.

Charge recovery using phases 30a and 30b is said to be “active” because the P/NDACs in stimulation circuitry 28 actively drive a current, in particular during the last phase 30b to recover charge stored after the first phase 30a. However, such active charge recovery may not be perfect, and some residual charge may be present in capacitive structures even after phase 30b is completed. Accordingly, the stimulation circuitry 28 can also provide for passive charge recovery. Passive charge recovery is implemented using passive charge recovery switches PRi 41 as shown in FIG. 3. These switches 41 when selected via assertion of control signals <Xi> couple each electrode node ei to a passive recovery voltage Vpr established on bus 43. As explained in U.S. Pat. Nos. 10,716,937 and 10,792,491, this allows any stored charge to be recovered through the patient's tissue, R. Control signals <Xi> are usually asserted to cause passive charge recovery after each pulse (e.g., after each last phase 30b) during periods 30c shown in FIG. 2A. Because passive charge recovery involves capacitive discharge through the resistance R of the patient's tissue, such discharge manifests as an exponential decay in current, as shown in FIG. 2A. As also discussed in the '937 patent, each of the passive charge recovery switches 41 can be associated with a variable resistance, and as such each switch 41 can be controlled by a bus of signals <Xi> to control the resistance at which passive charge recovery occurs—i.e., the on resistance of the switches 41 when they are closed. Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 27a in the IPG 10. The external controller 60 can also have a far-field RF antenna 64b capable of wirelessly communicating with the RF antenna 27b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 27a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 27b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

An increasingly interesting development in pulse generator systems, and in Spinal Cord Stimulator (SCS) pulse generator systems specifically, is the addition of sensing capability to complement the stimulation that such systems provide. FIG. 5 shows an IPG 100 that includes stimulation and sensing functionality. An ETS as described earlier could also include stimulation and sensing capabilities, and the circuitry shown in FIG. 5.

For example, it can be beneficial to sense a neural response in neural tissue that has received stimulation from the IPG 100. One such neural response is an Evoked Compound Action Potential (ECAP). An ECAP comprises a cumulative response provided by neural fibers that are recruited by the stimulation, and essentially comprises the sum of the action potentials of recruited neural elements (ganglia or fibers) when they “fire.” An ECAP is shown in isolation in FIG. 5, and comprises a number of peaks that are conventionally labeled with P for positive peaks and N for negative peaks, with P1 comprising a first positive peak, N1 a first negative peak, P2 a second positive peak, N2 a second negative peak, and so on. Note that not all ECAPs will have the exact shape and number of peaks as illustrated in FIG. 5, because an ECAP's shape is a function of the number and types of neural elements that are recruited and that are involved in its conduction. An ECAP is generally a small signal, and may have a peak-to-peak amplitude on the order of hundreds of microvolts or more.

FIG. 5 also shows an electrode array 17 comprising (in this example) a single percutaneous lead 15, and shows use of electrodes E3, E4 and E5 to produce pulses in a tripolar mode of stimulation, with (during the first phase 30a) E3 and E5 comprising anodes and E4 a cathode. Other electrode arrangements (e.g., bipoles, etc.) could be used as well. Such stimulation produces an electric field 130 in a volume of the patient's tissue centered around the selected electrodes. Some of the neural fibers within the electric field 130 will be recruited and fire, particularly those proximate to the cathodic electrode E4, forming ECAPs which can travel both rostrally toward the brain and caudally away from the brain. The ECAPs pass through the spinal cord by neural conduction with a speed which is dependent on the neural fibers involved in the conduction. In one example, the ECAP may move at a speed of about 5 cm/1 ms. U.S. Patent Application Publication 2020/0155019 describes a lead that can be useful in the detection of ECAPs.

ECAPs can be sensed at one or more sensing electrodes which can be selected from the electrodes 16 in the electrode array 17. Sensing preferably occurs differentially, with one electrode (e.g., S+, E8) used for sensing and another (e.g., S−, E9) used as a reference. This could also be flipped, with E8 providing the reference (S−) for sensing at electrode E9 (S+). Although not shown, the case electrode Ec (12) can also be used as a sensing reference electrode S−. Sensing reference S− could also comprise a fixed voltage provided by the IPG 100 (e.g., Vamp, discussed below), such as ground, in which case sensing would be said to be single-ended instead of differential.

The waveform appearing at sensing electrode E8 (S+) is shown in FIG. 5, which includes a stimulation artifact 134 as well as an ECAP. The stimulation artifact 134 comprises a voltage that is formed in the tissue as a result of the stimulation, i.e., as a result of the electric field 130 that the stimulation creates in the tissue. As described in U.S. Patent Application Publication 2019/0299006, the voltage in the tissue can vary between ground and the compliance voltage VH used to power the DACs, and so the stimulation artifact 134 can be on the order of Volts, and therefore significantly higher than the magnitude of stimulation-induced ECAPs. Generally speaking, the waveform sensed at the sensing electrode may be referred to as an ElectroSpinoGram (ESG) signal, which comprises the ECAP, the stimulation artifact 134, and other background signals that may be produced by neural tissue even absent stimulation. Realize that the ESG signal as shown at the sensing electrode S+ in FIG. 5 is idealized. The figures in U.S. Patent Application Publication 2022/0323764 show actual recorded ESG traces.

The magnitudes of the stimulation artifact 134 and the ECAP at the sensing electrodes S+ and S− are dependent on many factors, such as the strength of the stimulation, and the distance of sensing electrodes from the stimulation. ECAPs tend to decrease in magnitude at increasing stimulation-to-sensing distances because they disperse in the tissue. Stimulation artifacts 134 also decrease in magnitude at increasing stimulation-to-sensing distances because the electric field 130 is weaker at further distances. Note that the stimulation artifact 134 is also generally larger during the provision of the pulses, although it may still be present even after the pulse (i.e., the last phase 30b of the pulse) has ceased, due to the capacitive nature of the tissue or the capacitive nature of the driving circuitry (i.e., the DACs). As a result, the electric field 130 may not dissipate immediately upon cessation of the pulse.

It can be useful to sense in the IPG 100 features of either or both of the ECAPs or stimulation artifact 134 contained within the sensed ESG signal, because such features can be used to useful ends. For example, ECAP features can be used for feedback, such as closed-loop feedback, to adjust the stimulation the IPG 100 provides. Sec, e.g., U.S. Pat. No. 10,406,368 and U.S. Patent Application Publications 2019/0099602, 2019/0209844, 2019/0070418, 2020/0147393 and 2022/0347479. ECAP assessment can also be used to infer the types of neural elements or fibers that are recruited, which can in turn be used to adjust the stimulation to selectively stimulate such elements. Sec, e.g., U.S. Patent Application Publication 2019/0275331. Assessments of ECAP features can also be used to determine cardiovascular effects, such as a patient's heart rate. See, e.g., U.S. Patent Application Publication 2019/0290900. To the extent one wishes to assess features of an ECAP that are obscured by a stimulation artifact, U.S. Patent Application Publication 2019/0366094 discloses techniques that can used to extract ECAP features from the ESG signal. As discussed in some of these references, detected ECAPs can also be dependent on a patient's posture or activity, and therefor assessment of ECAP features can be used to infer a patient's posture, which may then in turn be used to adjust the stimulation that the IPG 100 provides.

It can also be useful to detect features of stimulation artifacts 134 in their own right. For example, U.S. Patent Application Publication 2022/0323764 describes that features of stimulation artifacts can be useful to determining patent posture or activity, which again may then in turn be used to adjust the stimulation that the IPG 100 provides.

FIG. 5 shows further details of the circuitry in an IPG 100 that can provide stimulation and sensing an ElectroSpinoGram (ESG) signal. The IPG 100 includes control circuitry 102, which may comprise a microcontroller, such as Part Number MSP430, manufactured by Texas Instruments, Inc., which is described in data sheets at http://www.ti.com/microcontrollers/msp430-ultra-low-power-mcus/overview.html, which are incorporated herein by reference. Other types of controller circuitry may be used in lieu of a microcontroller as well, such as microprocessors, FPGAs, DSPs, or combinations of these, etc. Control circuitry 102 may also be formed in whole or in part in one or more Application Specific Integrated Circuits (ASICs), such as those described and incorporated earlier.

The IPG 100 also includes stimulation circuitry 28 to produce stimulation at the electrodes 16, which may comprise the stimulation circuitry 28 shown earlier (FIG. 3). A bus 118 provides digital control signals from the control circuitry 102 (and possibly from an feature extraction algorithm 140, described below) to one or more PDACs 40; or NDACs 42; to produce currents or voltages of prescribed amplitudes (I) for the stimulation pulses, and with the correct timing (PW, F) at selected electrodes. As noted earlier, the DACs can be powered between a compliance voltage VH and ground. As also noted earlier, but not shown in FIG. 4, switch matrices could intervene between the PDACs and the electrode nodes 39, and between the NDACs and the electrode nodes 39, to route their outputs to one or more of the electrodes, including the conductive case electrode 12 (Ec). Control signals for switch matrices, if present, may also be carried by bus 118. Notice that the current paths to the electrodes 16 include the DC-blocking capacitors 38 described earlier, which provide safety by preventing the inadvertent supply of DC current to an electrode and to a patient's tissue. Passive recovery switches 41; (FIG. 3) could also be present, but are not shown in FIG. 5 for simplicity.

IPG 100 also includes sensing circuitry 115, and one or more of the electrodes 16 can be used to sense signals the ESG signal. In this regard, each electrode node 39 is further coupleable to a sense amp circuit 110. Under control by bus 114, a multiplexer 108 can select one or more electrodes to operate as sensing electrodes (S+, S−) by coupling the electrode(s) to the sense amps circuit 110 at a given time, as explained further below. Although only one multiplexer 108 and sense amp circuit 110 are shown in FIG. 5, there could be more than one. For example, there can be four multiplexer 108/sense amp circuit 110 pairs each operable within one of four timing channels supported by the IPG 100 to provide stimulation. The sensed signals output by the sense amp circuitry are preferably converted to digital signals by one or more Analog-to-Digital converters (ADC(s)) 112, which may sample the output of the sense amp circuit 110 at 50 kHz for example. The ADC(s) 112 may also reside within the control circuitry 102, particularly if the control circuitry 102 has A/D inputs. Multiplexer 108 can also provide a fixed reference voltage, Vamp, to the sense amp circuit 110, as is useful in a single-ended sensing mode (i.e., to set S− to Vamp).

So as not to bypass the safety provided by the DC-blocking capacitors 38, the inputs to the sense amp circuitry 110 are preferably taken from the electrode nodes 39. However, the DC-blocking capacitors 38 will pass AC signal components (while blocking DC components), and thus AC components within the ESG signals being sensed (such as the ECAP and stimulation artifact) will still readily be sensed by the sense amp circuitry 110. In other examples, signals may be sensed directly at the electrodes 16 without passage through intervening capacitors 38.

As noted above, it is preferred to sense an ESG signal differentially, and in this regard, the sense amp circuitry 110 comprises a differential amplifier receiving the sensed signal S+ (e.g., E8) at its non-inverting input and the sensing reference S− (e.g., E9) at its inverting input. As one skilled in the art understands, the differential amplifier will subtract S− from S+ at its output, and so will cancel out any common mode voltage from both inputs. This can be useful for example when sensing ECAPs, as it may be useful to subtract the relatively large scale stimulation artifact 134 from the measurement (as much as possible) in this instance. That being said, note that differential sensing will not completely remove the stimulation artifact, because the voltages at the sensing electrodes S+ and S− will not be exactly the same. For one, each will be located at slightly different distances from the stimulation and hence will be at different locations in the electric field 130. Thus, the stimulation artifact 134 can still be sensed even when differential sensing is used. Examples of sense amp circuitry 110, and manner in which such circuitry can be used, can be found in U.S. Patent Application Publications 2019/0299006, 2020/0305744, 2020/0305745 and 2022/0233866.

The digitized ESG signal from the ADC(s) 112—inclusive of any detected ECAPs and stimulation artifacts—is received at a feature extraction algorithm 140 programmed into the IPG's control circuitry 102. The feature extraction algorithm 140 analyzes the digitized sensed signals to determine one or more ECAP features, and one or more stimulation artifact features, as described for example in U.S. Patent Application Publication 2022/0323764. Such features may generally indicate the size and shape of the relevant signals, but may also be indicative of other factors (like ECAP conduction speed). One skilled in the art will understand that the feature extraction algorithm 140 can comprise instructions that can be stored on non-transitory machine-readable media, such as magnetic, optical, or solid-state memories within the IPG 100 (e.g., stored in association with control circuitry 102).

For example, the feature extraction algorithm 140 can determine one or more neural response features (e.g., ECAP features), which may include but are not limited to:
a height of any peak (e.g., N1);
a peak-to-peak height between any two peaks (such as from N1 to P2);
a ratio of peak heights (e.g., N1/P2);
a peak width of any peak (e.g., the full-width half-maximum of N1);
an area or energy under any peak;
a total area or energy comprising the area or energy under positive peaks with the area or energy under negative peaks subtracted or added;
a length of any portion of the curve of the ECAP (e.g., the length of the curve from P1 to N2);
any time defining the duration of at least a portion of the ECAP (e.g., the time from P1 to N2);
a time delay from stimulation to issuance of the ECAP, which is indicative of the neural conduction speed of the ECAP, which can be different in different types of neural tissues;
a conduction speed (i.e., conduction velocity) of the ECAP, which can be determined by sensing the ECAP as it moves past different sensing electrodes;
a rate of variation of any of the previous features, i.e., how such features change over time;
a power (or energy) determined in a specified frequency band (e.g., delta, alpha, beta, gamma, etc.) determined in a specified time window (for example, a time window that overlaps the neural response, the stimulation artifact, etc.);
any mathematical combination or function of these variables;

Such ECAP features may be approximated by the feature extraction algorithm 140. For example, the area under the curve may comprise a sum of the absolute value of the sensed digital samples over a specified time interval. Similarly, curve length may comprise the sum of the absolute value of the difference of consecutive sensed digital samples over a specified time interval. ECAP features may also be determined within particular time intervals, which intervals may be referenced to the start of simulation, or referenced from within the ECAP signal itself (e.g., referenced to peak N1 for example).

In this disclosure, ECAP features, as described above, are also referred to as neural features or neural response features. This is because such ECAP features contain information relating to how various neural elements are excited/recruited during stimulation, and in addition, how these neural elements spontaneously fired producing spontaneous neural responses as well.

The feature extraction algorithm 140 can also determine one or more stimulation artifact features, which may be similar to the ECAP features just described, but which may also be different to account for the stimulation artifact 134's different shape. Determined stimulation artifact features may include but are not limited to:
a height of any peak;
a peak-to-peak height between any two peaks;
a ratio of peak heights;
an area or energy under any peak;
a total area or energy comprising the area or energy under positive peaks with the area or energy under negative peaks subtracted or added;
a length of any portion of the curve of the stimulation artifact;
any time defining the duration of at least a portion of the stimulation artifact;
a rate of variation of any of the previous features, i.e., how such features change over time;
a power (or energy) determined in a specified frequency band (e.g., delta, alpha, beta, gamma, etc.) determined in a specified time window (for example, a time window that overlaps the neural response, the stimulation artifact, etc.);
any mathematical combination or function of these variables.

Again, such stimulation artifact features may be approximated by the feature extraction algorithm 140, and may be determined with respect to particular time intervals, which intervals may be referenced to the start or end of simulation, or referenced from within the stimulation artifact signal itself (e.g., referenced to a particular peak). Within this disclosure, any of the ECAP features, artifact features, and/or other recorded neural features are collectively referred to herein as “neural features.”

Once the feature extraction algorithm 140 determines one or more of these features, it may then be used to any useful effect in the IPG 100, and specifically may be used to adjust the stimulation that the IPG 100 provides, for example by providing new data to the stimulation circuitry 28 via bus 118. This is explained further in some of the U.S. patent documents cited above. For example, if the distance between the stimulation electrode(s) and the patient's spinal cord changes (for example, because of postural changes, coughing, movement, etc.), the stimulation may be adjusted based on the extracted features to maintain optimum therapeutic stimulation.

This introduction has focused primarily on using SCS for the treatment of pain. However, SCS may be used to treat other indications. For example, orthostatic hypotension (a sudden drop in blood pressure as a result of changing position) occurs in approximately six percent of individuals and is thought to be a predominant cause of falls and is also a risk factor for stroke and other cardiovascular disease. Orthostatic hypotension is typically treated pharmaceutically, meaning that the patient must be constantly medicated even though the problem only occurs under certain circumstances. Accordingly, there is a need in the art for non-pharmaceutical treatments for orthostatic hypotension.

### SUMMARY

Disclosed herein is a system for managing orthostatic hypotension in a patient having an implantable medical device comprising an implantable pulse generator (IPG) and one or more electrode leads, wherein each of the one or more electrode leads are configured for implantation in the patient's spinal column and comprise a plurality of electrodes configured to deliver electrical stimulation to the patient's spinal cord, the system comprising: control circuitry configured to: receive an indication of an actual or anticipated hypotensive event in the patient, and respond to the indication by causing stimulation circuitry of IPG to change from a first state to a second state, wherein: in the first state, the stimulation circuitry does not cause any of the electrodes to deliver to the patient's spinal cord any electrical stimulation configured to manage the patient's blood pressure, and in the second state, the stimulation circuitry causes one or more of the plurality of electrodes to deliver to the patient's spinal cord electrical stimulation that is configured to manage the patient's blood pressure. According to some embodiments, the control circuitry is configured within the IPG. According to some embodiments, the control circuitry is configured within an external controller configured to communicate with the IPG. According to some embodiments, the indication of an actual or anticipated hypotensive event comprises a signal from a blood pressure monitor indicating a drop in the patient's blood pressure. According to some embodiments, the blood pressure monitor is implantable in the patient. According to some embodiments, the indication of an actual or anticipated hypotensive event comprises a signal from an accelerometer. According to some embodiments, the accelerometer is configured within the IPG. According to some embodiments, the accelerometer is configured external to the patient. According to some embodiments, the signal from the accelerometer indicating an inertial change in the patient. According to some embodiments, the inertial change comprises the patient changing postures. According to some embodiments, the indication of an actual or anticipated hypotensive event is determined based on electric potentials recorded by one or more of the plurality of electrodes. According to some embodiments, the electric potentials are determined by: using the stimulation circuitry of the IPG to cause a first one or more of the plurality of electrodes to deliver to the patient's spinal cord first electrical stimulation that is configured to evoke the electric potentials and that is not configured to maintain the patient's blood pressure, using sensing circuitry of the IPG to cause a second one or more of the plurality of electrodes to record the electric potentials, and using control circuitry of the IPG to extract one or more features of the electric potentials. According to some embodiments, the one or more features are selected from the group consisting of an amplitude of any peak, an area under a curve, a curve length, and a difference between amplitudes of any two peaks. According to some embodiments, the indication of an actual or anticipated hypotensive event is determined by comparing the one or more features to one or more threshold values. According to some embodiments, the first stimulation is configured to manage a condition in the patient other than orthostatic hypotension. According to some embodiments, the indication of an actual or anticipated hypotensive event is indicative of a posture change of the patient. According to some embodiments, responding to the indication further comprises alerting the patient of the actual or anticipated hypotensive event. According to some embodiments, the control circuitry is configured to receive one or more indications that the patient has fallen. According to some embodiments, the indication that the patient has fallen is based on accelerometer signals. According to some embodiments, the control circuitry is configured to send an alert to the patient based on an indication that the patient has fallen. According to some embodiments, the control circuitry is configured to monitor for a patient response to the alert, and if no response is received, to send an alert to one or more remote locations. According to some embodiments, the electrical stimulation that is configured to maintain the patient's blood pressure comprises stimulation having a frequency of 25-70 Hz. According to some embodiments, the electrical stimulation that is configured to maintain the patient's blood pressure is delivered to the patients T8-L3 spinal level.

Also disclosed herein is a method for treating orthostatic hypotension in a patient having an implantable medical device comprising an implantable pulse generator (IPG) and one or more electrode leads, wherein each of the one or more electrode leads are configured for implantation in the patient's spinal column and comprise a plurality of electrodes configured to deliver electrical stimulation to the patient's spinal cord, the method comprising: receiving an indication of an actual or anticipated hypotensive event in the patient, and responding to the indication by causing stimulation circuitry of IPG to change from a first state to a second state, wherein: in the first state, the stimulation circuitry does not cause any of the electrodes to deliver to the patient's spinal cord any electrical stimulation configured to maintain the patient's blood pressure, and in the second state, the stimulation circuitry causes one or more of the plurality of electrodes to deliver to the patient's spinal cord electrical stimulation that is configured to maintain the patient's blood pressure. According to some embodiments, the indication of an actual or anticipated hypotensive event comprises a signal from a blood pressure monitor indicating a drop in the patient's blood pressure. According to some embodiments, the blood pressure monitor is implantable in the patient. According to some embodiments, the indication of an actual or anticipated hypotensive event comprises a signal from an accelerometer. According to some embodiments, the accelerometer is configured within the IPG. According to some embodiments, the accelerometer is configured external to the patient. According to some embodiments, the signal from the accelerometer indicates a that the patient is changing postures. According to some embodiments, the indication of an actual or anticipated hypotensive event is determined based on electric potentials recorded by one or more of the plurality of electrodes. According to some embodiments, the electric potentials are determined by: using the stimulation circuitry of the IPG to cause a first one or more of the plurality of electrodes to deliver to the patient's spinal cord first electrical stimulation that is configured to evoke the electric potentials and that is not configured to maintain the patient's blood pressure, using sensing circuitry of the IPG to cause a second one or more of the plurality of electrodes to record the electric potentials, and using control circuitry of the IPG to extract one or more features of the electric potentials. According to some embodiments, the one or more features are selected from the group consisting of an amplitude of any peak, an area under a curve, a curve length, and a difference between amplitudes of any two peaks. According to some embodiments, the indication of an actual or anticipated hypotensive event is determined by comparing the one or more features to one or more threshold values. According to some embodiments, the first stimulation is configured to provide pain relief to the patient. According to some embodiments, the indication of an actual or anticipated hypotensive event is indicative of a posture change of the patient. According to some embodiments, the indication further comprises alerting the patient of the actual or anticipated hypotensive event. According to some embodiments, the method further comprises monitoring for one or more indications that the patient has fallen. According to some embodiments, the indication that the patient has fallen is based on accelerometer signals. According to some embodiments, if an indication that the patient has fallen is detected, the method further comprises sending an alert to the patient. According to some embodiments, the method further comprises monitoring for a patient response to the alert, and if no response is received, to send an alert to one or more remote locations. According to some embodiments, the electrical stimulation that is configured to maintain the patient's blood pressure comprises stimulation having a frequency of 25-70 Hz. According to some embodiments, the electrical stimulation that is configured to maintain the patient's blood pressure is delivered to the patients T8-L3 spinal level.

Also disclosed herein is a method for treating orthostatic hypotension in a patient having an implantable medical device comprising an implantable pulse generator (IPG) and one or more electrode leads, wherein each of the one or more electrode leads are configured for implantation in the patient's spinal column and comprise a plurality of electrodes configured to deliver electrical stimulation to the patient's spinal cord, the method comprising: using a first one or more of the electrodes to apply first electrical stimulation to the patient's spinal cord, wherein the first stimulation is configured to treat pain in the patient and is not configured to maintain the patient's blood pressure, receiving an indication of an actual or anticipated hypotensive event in the patient, and in response to the indication, using a second one or more of the electrodes to apply second electrical stimulation to the patient's spinal cord, wherein the second stimulation is configured to maintain the patient's blood pressure.

### BRIEF DESCRIPTION OF THE DRAWINGS

FIG. 1 shows an Implantable Pulse Generator (IPG).

FIGS. 2A and 2B show an example of stimulation pulses producible by the IPG.

FIG. 3 shows stimulation circuitry useable in the IPG.

FIG. 4 shows various external devices capable of communicating with and programming stimulation in an IPG.

FIG. 5 shows an IPG having neural response sensing capability.

FIG. 6 shows a system for monitoring and managing orthostatic hypotension.

FIG. 7 shows configurations of an electrode lead with respect to a patient's spinal cord as the patient changes postures.

### DETAILED DESCRIPTION

Aspects of the disclosure relate to methods and systems for applying SCS to manage a patient's blood pressure, for example, in the context of orthostatic hypotension. The management may comprise applying stimulation to attenuate, mitigate avoid, or slow down a drop in the patient's blood pressure when certain events occur. The events may include a detected drop in blood pressure, a change in posture, another inertial change, such as a fall or abrupt change in direction, a detected arrythmia, and the like. A benefit of the disclosed methods and systems compared to pharmaceutical treatment is that the disclosed methods and systems monitor for changes in blood pressure and/or changes in the patient's posture, and apply stimulation only when it is needed. The disclosed methods and systems may be used for patients with, or without spinal cord injury. For example, they may be implemented in a patient that already has an implanted SCS system to treat other indications, such as pain. Likewise, the disclosed methods and systems may be employed as a bespoke management for severe orthostatic hypotension, even absent other conditions typically treated using SCS.

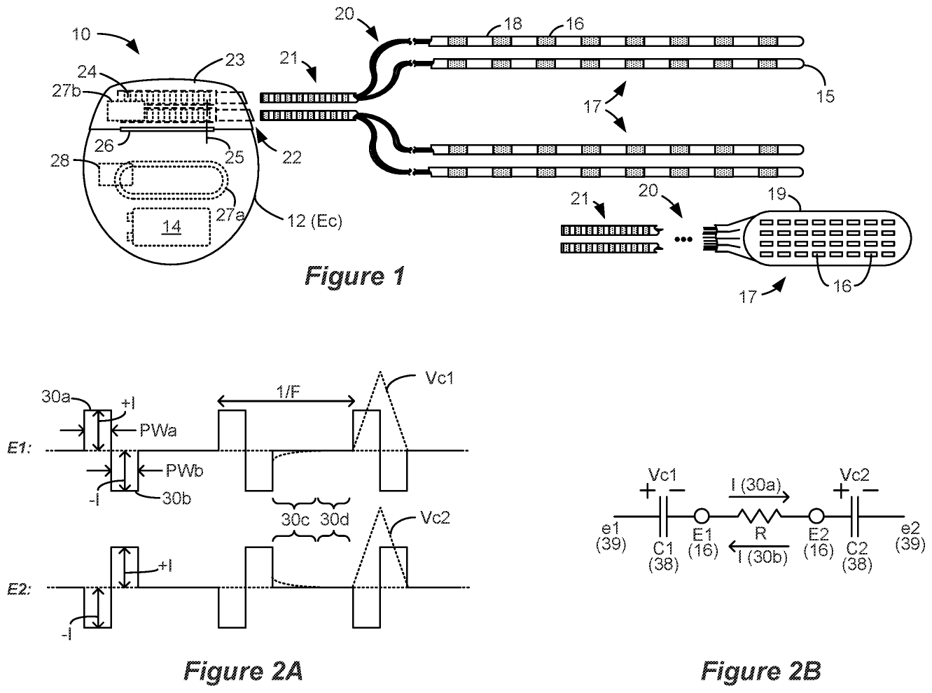

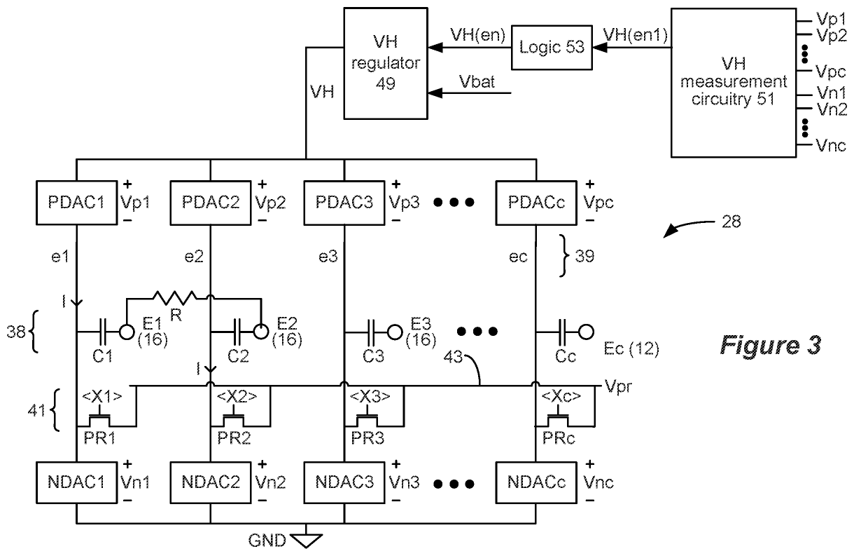

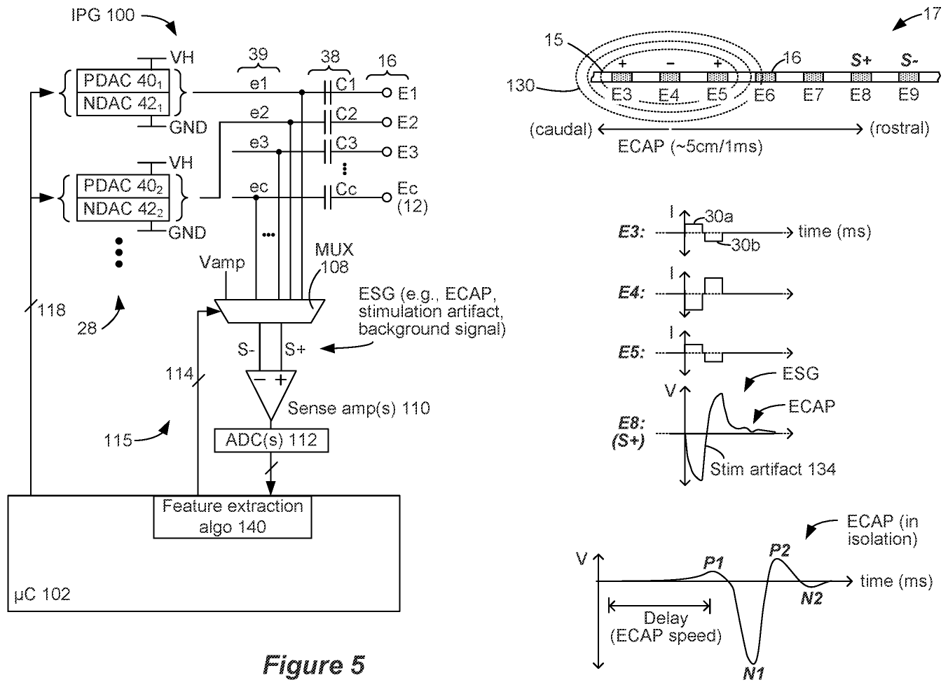

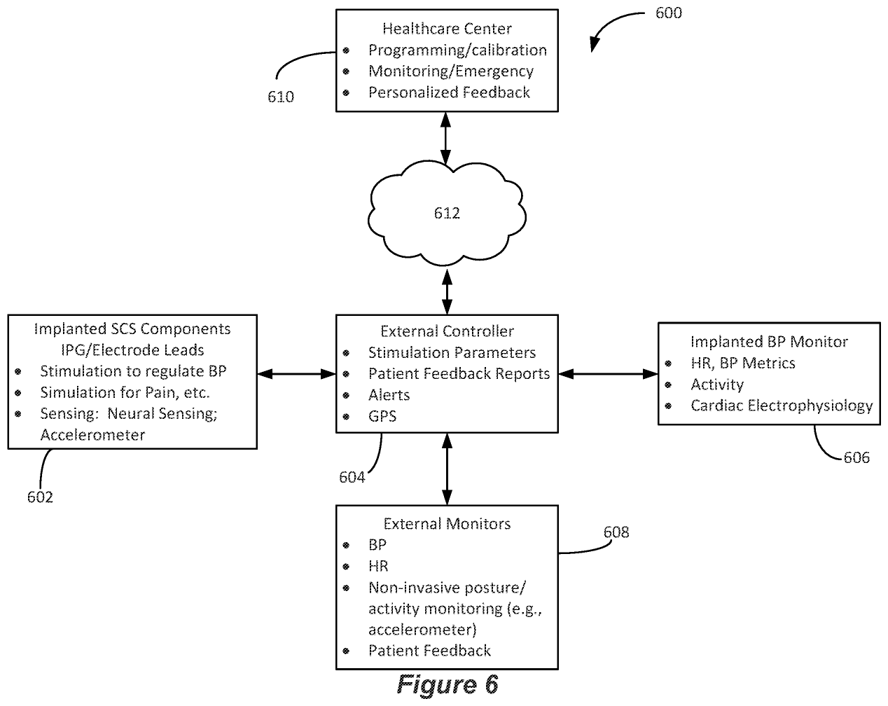

FIG. 6 illustrates an embodiment of a system 600 according to some embodiments the disclosure. The system comprises implanted SCS components 602. The SCS components comprise an IPG, such as the IPG 10 (FIG. 1) described above. Likewise, the SCS components comprise electrode leads (e.g., leads 15/19, FIG. 1). The SCS components are configured to be implanted in a patient, as described above. Specifically, for modulating orthostatic hypotension, the electrode leads may be implanted within the patient's spinal column and configured to provide stimulation between the lower thoracic and upper lumbar spinal levels (e.g., ˜T8-L3 spinal levels). As mentioned above, the IPG may also be configured for providing stimulation to address other indications, such as pain. According to some embodiments, the IPG may be configured to provide stimulation in the 25-70 Hz range. According to some embodiments, the IPG may also be configured for sensing, as described above with reference to FIG. 5. According to some embodiments, the IPG may also be configured with one or more accelerometers for measuring data indicative of the patient's activity and/or posture. The use of activity/posture measurements will be discussed in more detail below.

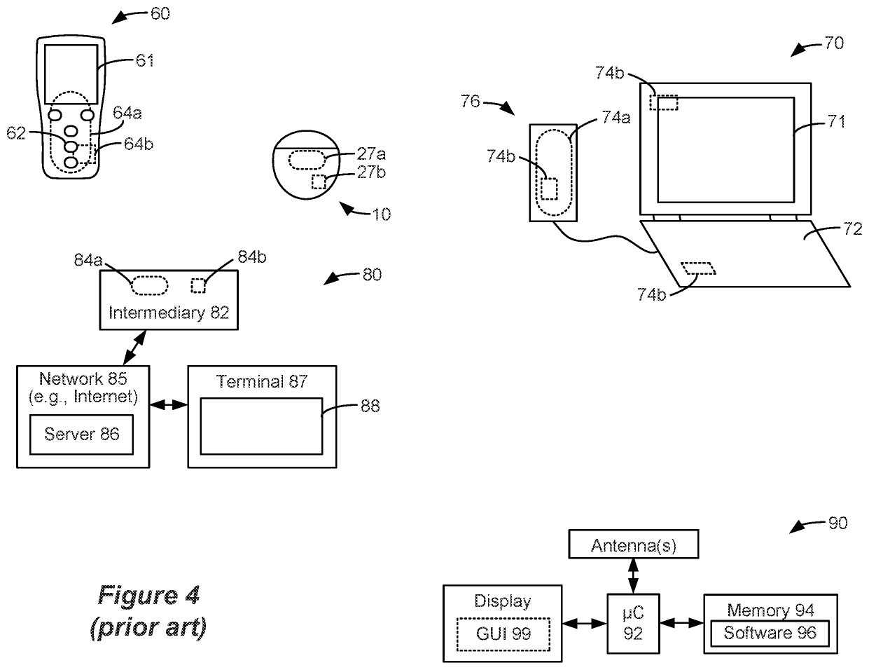

The system 600 also includes an external controller 604 that the patient may use to control their stimulation. The external controller 604 may include the functionality described above with respect to the external controller 60 (FIG. 4). For example, the patient can use the external controller to turn the stimulation on and off and to modify stimulation parameters. The external controller 604 may be configured with telemetry capabilities for communicating stimulation parameters to (and receiving sensing data from) the implanted components 602, as described above. The external controller 604 may be configured for telemetry with other components of the system 600, as described in more detail below.

Embodiments of the system 600 may also comprise an implanted blood pressure monitor 606. The blood pressure monitor may be configured to monitor blood pressure, heart rate, cardiac electrophysiology, etc. Examples of implantable blood pressure monitors may include arterial pressure sensors, heart sound sensors, and/or any blood pressure monitor known in the art. The implantable blood pressure monitor may be temporarily implantable, permanent, or semi-permanent. According to some embodiments, the implantable blood pressure monitor may be configured to communicate with the external controller 604.

Embodiments of the system 600 may also comprise non-invasive/external monitors 608. Examples of such external monitors include wearable devices, such as wearable blood pressure monitors, heartrate monitors, accelerometers, and the like. Examples of wearables include watches, such as smart watches. The external monitors may also provide user interfaces to allow the patient to provide input, such as feedback regarding their activity, health state, etc. According to some embodiments the external monitors may communicate with the external controller 604.

Embodiments of the 600 may also comprise a remotely located monitoring center 610 that may connect to the other system components via an internet 612 connection, for example. According to some embodiments, a Wi-Fi and/or cellular-equipped external controller 604 may communicate with the remote monitoring center (e.g., healthcare center, etc.) via the internet connection. Actions performed by the remote monitoring center will be discussed in more detail below. It should be noted that the external controller serves as a hub in the system 600 illustrated in FIG. 6. In other words, each of the system components communicate with each other via the external controller. However, in other embodiments, some or all of the system components may communicate with each other without the intervening external controller. For example, the blood pressure monitor may communicate with the IPG of the implanted SCS components, etc.

As mentioned above, the system 600 may be configured to provide stimulation to the patient in response to a measured, or predicted, drop in the patient's blood pressure. According to some embodiments, the implanted blood pressure monitor 606 (or an external blood pressure monitor) may sense a drop in blood pressure and may initiate the system to provide stimulation. For example, the implanted (or external) blood pressure monitor may communicate the need for stimulation to the external controller 604. According to some embodiments, the external controller may issue an alert to the patient indicating the need for stimulation. The alert may be an alarm, vibration, or text message, for example, prompting the patient to activate stimulation. According to other embodiments, the external controller may automatically activate stimulation with or without informing the patient. Alternatively, the blood pressure monitor may communicate directly with the implanted SCS components to activate stimulation. According to some embodiments, the system may compare the measured blood pressure (or a rate of change of the blood pressure) to a threshold value to determine if stimulation is warranted. For example, the patient's external controller may comprise an algorithm for making that determination based on threshold values. For example, the blood pressure monitor may activate stimulation for a patient with known orthostatic hypotension after a detection of a drop of 20 mm Hg of systolic blood pressure or a 10 mm Hg of diastolic blood pressure. If the patient has known hypertension the thresholds could occur at higher thresholds. According to some embodiments, the system may adapt to previously recorded orthostatic hypotensive events and the response to stimulation. This may be used to shift to an automatic activation of stimulation at lesser thresholds or initiate activation at higher amplitudes (within the clinician set threshold) then initially set According to some embodiments, the system may monitor for a difference from a patient-specific baseline upon registering an event, or difference from a healthy reference value upon registering an event. Example events include a fall, sign of exercise, or other abrupt change in movement, an indication of arrythmia, etc. According to some embodiments, the stimulation may be continued until the blood pressure stabilizes. According to other embodiments, the stimulation may be provided for a predetermined amount of time, e.g., 10 minutes. According to some embodiments, the stimulation may be cycled on and off, for example, 5 seconds on and 5 seconds off (or different durations) to enable BP detection after stimulation epochs. If the blood pressure stabilizes after the first epoch, then the off portion of the stimulation can be lengthened, for example, in increments.

According to some embodiments, the blood pressure monitor may continue to monitor the patient's blood pressure to determine if the stimulation is effective, i.e., to determine if the patient's blood pressure is stabilized. In the event that the blood pressure is not stabilized and continues to drop, the patient's external controller may issue a notice/warning to the patient that their blood pressure is continuing to drop, thereby prompting them to take an action, such as sitting/lying down, and/or seeking medical attention. According to some embodiments, the system may be configured to adjust the stimulation (for example, increase the amplitude of stimulation) if the initial stimulation is not effective at stabilizing the patient's blood pressure.

According to some embodiments, sensors other than the blood pressure monitor may be used to monitor the patient's state and predict when stimulation may be needed. These other sensors are referred to herein as “secondary sensors.” Such secondary sensors may be used to support and augment the direct blood pressure measurements, or they may be used as a proxy for the blood pressure measurements in embodiments of the system that do not include a direct blood pressure monitor.

One example of such secondary sensors includes inertial sensors, such as accelerometers, gyroscopes, magnetometers, and the like. As used herein, the term “accelerometer” will be understood to refer to any type of inertial sensor. According to some embodiments, the IPG of the implanted SCS components may comprise one or more accelerometers. According to other embodiments, the accelerometer(s) may be external, for example, a component of the patient's external controller (smart phone) and/or as a component of a wearable device (e.g., watch or smartwatch). Data from the one or more accelerometers may be analyzed to determine when the patient changes positions, e.g., when they stand up from a sitting or lying down posture. According to some embodiments, data from the accelerometer(s) may be processed to determine an acceleration as the patient changes positions. According to some embodiments, the rate of an acceleration change (dA/dt, i.e., “jerk”) may be determined. The acceleration and/or jerk values may be compared to a threshold value to evaluate the need for stimulation. The threshold values may be determined based on patient-specific calibration (either against hypotensive symptoms or of blood pressure drop vs. “jerk” vs. position). According to some embodiments, the stimulation may be maintained as long as the patient is in an upright positions. According to some embodiments, the stimulation may be maintained for a period of time (e.g., ten minutes). The patient may be prompted whether or not to continue stimulation at the end of the stimulation period. According to some embodiments, the stimulation may be processed to determine if the patient is moving in a vehicle (for example, the acceleration in a lateral direction is high), in which case, the stimulation may be turned off. According to some embodiments, the accelerometer data may be processed to distinguish between standing and sitting positions. For example, if there is no lateral movement for a given period of time (e.g., ten minutes), it may be assumed that the patient is sitting, and the stimulation may be turned off.

Another embodiment of secondary sensed information for predicting the need for stimulation may comprise posture/activity determinations derived from sensed neural signals in the patient's spinal column, such as the ECAPs mentioned above. As explained above, the intensity and/or morphology of sensed ECAPs may be posture/activity dependent. This is because the amount of cerebrospinal fluid between the electrodes and the patient's spinal tissue changes when the patient changes postures. U.S. Patent Publication No. 2022/0266027, the contents of which are incorporated herein by reference, describes how postural changes may be reflected in sensed ECAP signals.

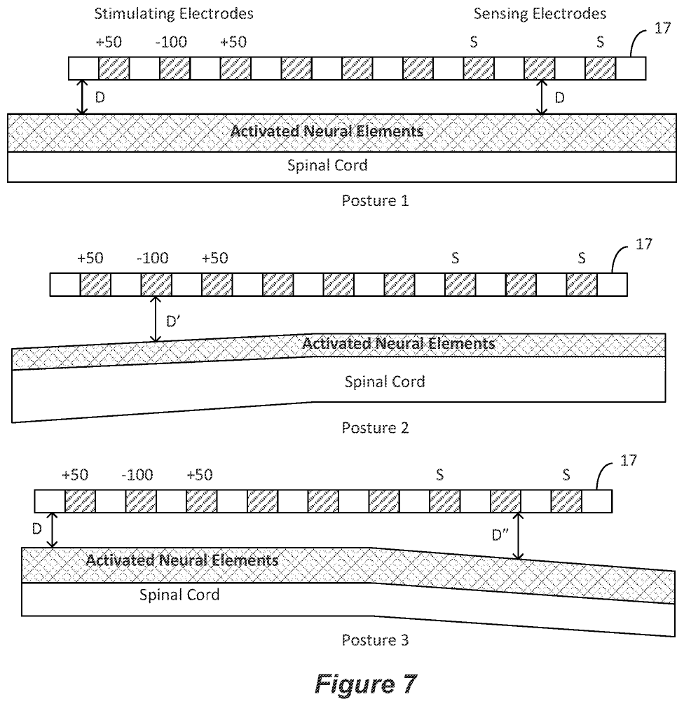

Briefly, FIG. 7 shows different configurations of an electrode lead with respect to the patient's spinal cord when the patient is in different postures. Notice that when the patient is Posture 1, the stimulating electrodes and the sensing electrodes are each a distance D from the spinal cord. The stimulation at that distance will activate a certain number of neural elements within the patient's spinal cord. The activated neural elements will elicit a neural response (e.g., an ECAP), which may be sensed at the illustrated sensing electrodes(S). When the patient changes postures to Posture 2, the stimulating environment changes. Specifically, the stimulating electrodes are further from the patient's spinal cord (new distance D′) because the spinal cord has moved. The change in the distance between the stimulating electrodes and the spinal cord causes an increase in the thickness of the cerebrospinal fluid (dCSF) between the stimulating electrodes and the target neural elements. Since the stimulating electrodes are further from the spinal cord, the stimulation may activate fewer neural elements. Since fewer neural elements are activated, the magnitude of sensed neural response features will decrease, and the line-shape of the sensed neural response may change. Accordingly, changes in the morphology of the sensed neural response may be used to indicate a change in posture. According to some embodiments, the system may be configured to sense the neural responses (e.g., ECAPs) and to extract one or more features of the neural response to monitor as potential indicators of postural changes. Examples of such features include neural response amplitudes, and/or other intensity metrics (such as the area under the curve (AUC), curve length, etc.).

As another example, when the patient changes to Posture 3, the stimulating electrodes are still at the original calibrated distance D from the spinal cord. Thus, the same amount of neural elements are activated as in Posture 1. However, the distance between the sensing electrodes and the spinal cord is increased because of movement of the spinal cord (new distance D″). Thus, the magnitude of the sensed neural response is decreased because there is more dCSF between the sensing electrodes and the spinal cord, even though the same amount of neural elements are activated by the stimulation. Again, such postural changes may be detected based on extracted features of the measured neural response.

Aspects of this disclosure involve using sensed neural responses and/or sensed stimulation artifact features as an indication of postural changes to trigger stimulation for maintaining the patient's blood pressure. Essentially, any of the aspects or features of sensed neural potentials that are described above for use for closed loop feedback for maintaining pain controlling stimulation may also be leveraged for indicating postural changes (thereby signaling the need for blood pressure-maintaining stimulation). According to some embodiments, the patient may be receiving SCS stimulation for maintaining pain or for some other indication like a gait and/or balance deficit. A sensed neural response to that stimulation may indicate a postural change and trigger blood pressure-maintaining stimulation. According to other embodiments, stimulation may be issued to the patient for the bespoke purpose of monitoring evoked neural responses to detect postural changes.

According to some embodiments, the clinician may perform a calibration routine with the patient whereby the patient changes postures and the clinician notes which evoked neural features are good indicators of postural changes that may lead to orthostatic hypotensive events. The system can be configured to monitor for such indicators and use those indicators as a trigger to provide stimulation for regulating the patient's blood pressure. For example, either the IPG of the implanted SCS components (602, FIG. 6) and/or the external controller (604, FIG. 6) may comprise one or more algorithms configured to extract one or more features of recorded evoked potentials in the patient's spinal cord/spinal column to use as triggers. According to some embodiments, the extracted neural features may be compared to one or more threshold values (or ranges).

According to some embodiments, either the IPG of the implanted SCS components (602, FIG. 6) and/or the external controller (604, FIG. 6) and/or the remote monitoring center (610, FIG. 6) may comprise one or more algorithms configured to adaptively correlate one or more extracted features of recorded neural signals with blood pressure changes determined, for example, from implanted or external blood pressure monitors. According to some embodiments, either the IPG of the implanted SCS components (602, FIG. 6) and/or the external controller (604, FIG. 6) and/or the remote monitoring center (610, FIG. 6) may comprise one or more algorithms configured to adaptively correlate one or more extracted features of recorded neural signals with postural changes, for example, indicated by accelerometer data.

Here we note that the use of extracted features of neural responses (i.e., ECAP features, stimulation artifact features, and the like) as a means of detecting postural changes for triggering stimulation to regulate the patient's blood pressure (as described here) may be distinguished from the use of neural responses for closed loop feedback for adjusting stimulation (as discussed in the Introduction above). Embodiments of the disclosed methods may use closed loop feedback based on features of recorded neural responses. But this particular discussion concerns using extracted features of evoked neural responses to indicate postural changes that indicate the need for blood pressure-controlling stimulation. In other words, the neural responses are used to detect the need for stimulation and to trigger the activation of stimulation. For example, the neural responses may be used to trigger the system to switch from a first state, during which no blood pressure-maintaining stimulation is being provided, to a second state, during which blood pressure-managing stimulation is provided to prevent or treat a hypotensive event.

We also note that an advantage of embodiments the described systems and methods is that therapy (either pharmaceutical or electrical stimulation therapy) is not provided on a continuing basis (i.e., chronically). Instead, the system may remain in a first state, during which blood pressure-maintaining therapy is not being provided until a triggering event is detected. The triggering event may be a drop in blood pressure detected using an implanted or external monitor, an accelerometer reading, a postural change indicated based on sensed evoked neural responses, or the like. When the trigger is detected, the system may switch to a second state, during which blood pressure-maintaining stimulation is provided for as long as needed. The system may then switch back to the first state.

According to some embodiments, the system may be configured so that the patient can toggle the sensing and stimulation functionality on and off. For example, if the patient is going to exercise or participate in some other activity that is likely to cause acceleration and/or postural triggers, the patient may elect to turn off the system's sensing capabilities to avoid unwanted stimulation triggering.

Referring again to FIG. 6, it was mentioned above that the system 600 may be configured for communication between the patient's external controller and a remote monitoring center 610. According to some embodiments, the patient's external controller may send the remote monitoring center information concerning when and how the system is being used. For example, the external controller may send information indicating when and how often the stimulation is being triggered to provide stimulation and/or when the patient is proactively selecting to initiate stimulation. According to some embodiments, one or more algorithms may be used to correlate patient-initiated stimulation with other system measurements, such as detected posture changes, accelerometer measurements, implantable blood pressure measurements, and the like. One example of such algorithms includes machine learning/adaptive learning algorithms that can be trained to predict the need for stimulation based on the measured indicators and to cause the stimulator to automatically apply stimulation under appropriate circumstances. Such learning algorithms may be configured at the remote monitoring center and/or they may be configured within the external controller 604. According to some embodiments, the information transmitted to the remote monitoring center may be used to provide the patient with personalized feedback concerning their orthostatic hypotension and their stimulator use and/or personalized programming for the operation of the system.

According to some embodiments, the system may be configured for monitoring for emergency situations. For example, if the system detects an abrupt drop in blood pressure, which may be indicative of a syncope event, and/or an abrupt acceleration, which may be indicative of the patient falling, that information may be transmitted to a remote location, such as a monitoring center and/or to one or more non-medical parties such as a caregiver, family member, or friend. In response, the monitoring center may send an alert/message to the patient asking the patient to confirm that they are okay. The remote monitoring facility may be configured to alert emergency personnel and/or the patient's emergency contacts if no reply is received. According to some embodiments, the patient's external controller may be configured with GPS capabilities, enabling the emergency personnel to respond to the patient's location. According to some embodiments, the patient's external controller may be configured to perform the emergency monitoring automatically without involvement of the remote monitoring center.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for managing orthostatic hypotension in a patient having an implantable medical device comprising an implantable pulse generator (IPG) and one or more electrode leads, wherein each of the one or more electrode leads are configured for implantation in the patient's spinal column and comprise a plurality of electrodes configured to deliver electrical stimulation to the patient's spinal cord, the system comprising:
control circuitry configured to:
receive an indication of an actual or anticipated hypotensive event in the patient, and
respond to the indication by causing stimulation circuitry of IPG to change from a first state to a second state, wherein:
in the first state, the stimulation circuitry does not cause any of the electrodes to deliver to the patient's spinal cord any electrical stimulation configured to manage the patient's blood pressure, and
in the second state, the stimulation circuitry causes one or more of the plurality of electrodes to deliver to the patient's spinal cord electrical stimulation that is configured to manage the patient's blood pressure.

2. The system of claim 1, wherein the indication of an actual or anticipated hypotensive event comprises a signal from a blood pressure monitor indicating a drop in the patient's blood pressure.

3. The system of claim 1, wherein the blood pressure monitor is implantable in the patient.

4. The system of claim 1, wherein the indication of an actual or anticipated hypotensive event comprises a signal from an accelerometer.

5. The system of claim 3, wherein the accelerometer is configured within the IPG.

6. The system of claim 3, wherein the accelerometer is configured external to the patient.

7. The system of claim 4, wherein the signal indicates a posture change of the patient.

8. The system of claim 1, wherein the indication of an actual or anticipated hypotensive event is determined based on electric potentials recorded by one or more of the plurality of electrodes.

9. The system of claim 8, wherein the electric potentials are determined by:
using the stimulation circuitry of the IPG to cause a first one or more of the plurality of electrodes to deliver to the patient's spinal cord first electrical stimulation that is configured to evoke the electric potentials and that is not configured to maintain the patient's blood pressure,
using sensing circuitry of the IPG to cause a second one or more of the plurality of electrodes to record the electric potentials, and
using control circuitry of the IPG to extract one or more features of the electric potentials.

10. The system of claim 9, wherein the one or more features are selected from the group consisting of an amplitude of any peak, an area under a curve, a curve length, and a difference between amplitudes of any two peaks.

11. The system of claim 10, wherein the indication of an actual or anticipated hypotensive event is determined by comparing the one or more features to one or more threshold values.

12. The system of claim 9, wherein the first stimulation is configured to manage a condition in the patient other than orthostatic hypotension.

13. The system of claim 8, wherein the indication of an actual or anticipated hypotensive event is indicative of a posture change of the patient.

14. The system of claim 1, wherein responding to the indication further comprises alerting the patient of the actual or anticipated hypotensive event.

15. The system of claim 1, wherein the control circuitry is configured to receive one or more indications that the patient has fallen.

16. The system of claim 15, wherein the indication that the patient has fallen is based on accelerometer signals.

17. The system of claim 15, wherein the control circuitry is configured to send an alert to the patient based on an indication that the patient has fallen.

18. The system of claim 17, wherein the control circuitry is configured to monitor for a patient response to the alert, and if no response is received, to send an alert to one or more remote locations.

19. The system of claim 1, wherein the electrical stimulation that is configured to maintain the patient's blood pressure comprises stimulation having a frequency of 25-70 Hz.

20. The system of claim 1, wherein the electrical stimulation that is configured to maintain the patient's blood pressure is delivered to the patients T8-L3 spinal level.
